(19) United States
(12) Patent Application Publication
(10) Pub. No.: US 2022/0110526 A1
(43) Pub. Date: Apr. 14, 2022

(54) SYSTEM AND METHOD FOR FLUORESCENCE IMAGING OF BIOLOGICAL TISSUES

(71) Applicant: SPRING VISION LTD., Haifa (IL)

(72) Inventors: Avihu Meir GAMLIEL, Pardes-Hana (IL); Noam ALLON, Haifa (IL); Michael ARONOV, Haifa (IL); Eyal MARGALIT, Atzmon-Segev (IL)

(21) Appl. No.: 17/556,644

(22) Filed: Dec. 20, 2021

Related U.S. Application Data
(63) Continuation-in-part of application No. PCT/IL2020/050691, filed on Jun. 21, 2020.

(30) Foreign Application Priority Data
Jun. 20, 2019 (US) 62864191

(51) Int. Cl. A61B 5/00; A61B 3/12; G01N 21/64

(52) U.S. Cl. CPC A61B 5/0071; G01N 21/6456; A61B 3/12

(57) ABSTRACT

System and method are presented for use in inspecting a biological tissue. According to this technique, a predetermined location within the biological tissue is illuminated by an illumination pattern comprising a predetermined exciting wavelength range selected to substantially concurrently induce two or more auto-fluorescent responses of two or more predetermined different biological substances of types naturally existing in biological tissues to enable detection of a combined spectral response of the biological tissue to said illumination pattern. Upon identifying in said detected combined spectral response emission wavelengths of said two or more substances, output data is generated being indicative of simultaneous existence on said location of the combination of said two or more predetermined different biological substances, which provides direct indication about said pathological condition.

### CROSS REFERENCE TO RELATED APPLICATION

This application is a by-pass continuation-in-part application of International Application No. PCT/IL2020/050691 filed Jun. 21, 2020 claiming priority from U.S. Provisional Patent Application No. 62/864,191 filed Jun. 20, 2019. The entire contents of the international application and the priority application are incorporated herein by reference.

### TECHNOLOGICAL FIELD

The invention relates to imaging of biological tissues and is specifically relevant to imaging using auto-fluorescence response of biological substances in tissues.

### BACKGROUND

Various techniques are known for imaging biological samples. While the typical imaging techniques rely on reflection or transmission properties of biological materials, some applications utilize imaging of fluorescent response from the tissue. The use of fluorescent response enables improved detection of selected materials and provides additional information related to biological and/or medical conditions of the inspected tissue. These techniques may be directed for determining one or more selected biological materials for providing data relevant to selected parameters to be inspected.

There are various fluorescent imaging techniques known in the field of medical and biological imaging. Such techniques include, for example, Fluorescein Angiography (FA), Fundus Auto fluorescence (FAF) and others.

Fluorescein Angiography (FA) is currently a preferred method for imaging of the retinal microcirculation. This technique is used to detect many pathological processes in the retina, including leakages from blood vessels, development of edema and retinal vessel occlusions. Generally, FA requires the use of a dedicated fundus camera equipped with excitation and barrier filters and a selected fluorescent agent. To provide fluorescent response, fluorescein dye is injected intravenously, usually through an antecubital vein with sufficient speed to produce high contrast images of the early phases of the angiogram. White light from a flash is passed through a blue excitation filter. Blue light (wavelength 465-490 nm) is then absorbed by unbound fluorescein molecules, and the molecules emit fluorescent light with a wavelength in the yellow-green range of the visible spectrum (520-530 nm). A barrier filter of 520-530 nm allows capturing light emitted only from the excited fluorescein. Images are acquired immediately after injection and are continued for up to ten minutes depending on the pathology and test requirements.

A wide range of complications can occur with FA. The most common reactions are transient nausea that occurs in 3-15% of patients, vomiting (7%), and pruritus. More severe reactions such as urticaria, fever, thrombophlebitis, and syncope are rarer. Local tissue necrosis can occur with extravasation of dye; however, mild pain and redness are more typical. Severe life-threatening reactions such as anaphylaxis, cardiac arrest, and bronchospasm do occur but are extremely rare. Death is estimated to occur in 1:221,781. No serious adverse events have been reported to occur in pregnancy, however it is considered a contraindication.

Fundus Auto fluorescence (FAF) is a non-invasive imaging technique that detects various naturally occurring fluorophores. Classically, FAF utilizes blue-light excitation, and collects emissions within a preset spectrum to form a brightness map reflecting the distribution of lipofuscin, a dominant fluorophore located in the Retinal pigment epithelium (RPE). FAF may use other excitation wavelengths to detect additional fluorophores, such as near-infrared for detection of melanin auto-fluorescence. First described by Delori in the 1980s, FAF has since expanded in both scope and practice. Given the unique findings not identified with funduscopic examination, fundus photography, or fluorescein angiography, FAF has been useful in the evaluation of a diverse spectrum of diseases involving the retina and RPE, including degenerative, dystrophic, inflammatory, infectious, neoplastic, and toxic etiologies. This review summarizes the known ocular fluorophores, various imaging modalities, and broad clinical applications of FAF.

### GENERAL DESCRIPTION

There is a need in the art for a novel imaging technique enabling detection of one or more biological auto-fluorescent spectral agents (BASAs) in biological systems.

Auto-fluorescence in the human body is based on visualizing various constituents of different tissues, when the specific substance of choice (fluorophore) is illuminated and excited by UV, deep blue or NIR spectral range of electromagnetic radiation. In general, auto-fluorescence can be achieved in a wide range of substances, including albumin, elastin, collagen, lipofuscin, fatty acids, LDL, NADH, flavins, porphyrins and potentially others. Detection of such materials in different biological tissues may be associated with unusual material concentrations, e.g. due to leakage of various substances from capillaries and from damaged cells that undergo lysis under conditions of disrupted cellular metabolism. Such leakages can be of intracellular, as well as extracellular, origin and form sediments in the tissue itself, which enables to visualize them. For the purposes of the present application, all such substances are referred to as biological auto-fluorescent spectral agents (BASAs).

Thus, the present invention utilizes auto-fluorescent properties of selected BASAs, that naturally exist in biological systems, in order to assist in diagnosis of various pathologies. The invention is based on the inventors' understanding that detection (substantially simultaneous detection) of the simultaneous presence of a predetermined plurality (two or more) of BASA-relating materials/substances in a region/location within a biological tissue where such BASAs do not naturally accumulate all together, and/or detection (substantially simultaneously) of such BASAs accumulating in higher than normal amounts, enables diagnosis of various pathologies of the biological tissue.

More specifically, the inspection technique is intended to be performed on eye tissues (such as retina, sclera, cornea and eye lens) because of the optical transparency of the eye tissues enabling imaging of blood vessels. The technique of the invention thus also takes advantage of the optical properties of the eye tissues for detection of the presence of BASAs in their physiological amounts and configuration in certain tissues (like in blood-vessel walls) while providing enhanced contrast of those particular tissues, compared to their surroundings.

Auto-fluorescent properties of various BASAs are generally known. For example, it is known that human blood plasma contains several fluorophores, including albumin, Nicotinamide, Adenine dinucleotide (NADH), Flavin, Adenine dinucleotide (FAD), fatty acids and endogenous porphyrins and their derivatives. Albumin is known to be the most abundant substance in the human plasma, comprising roughly 50% of the plasma volume. Human plasma albumin has an excitation peak at about 280 nm and emission maximum at 330-350 nm. Bovine serum excitation in the range 340-400 results in emission at 450-550 nm. Human plasma has an excitation maximum at 400-420 nm and emission maximum at 460-520 nm. Lipids, lipoproteins, NADH and FAD are potential retinal fluorophores which can be used for auto-fluorescence imaging in pathological conditions such as diabetic retinopathy. Low density lipoprotein (LDL) has an absorption maximum at 282 nm and emission maximum at 331 nm. Fatty acids, the constituents of lipids, have absorption maximum at 330-350 nm and emission maximum at 470-480 nm. NADH has an absorption maximum at 340-380 nm and emission maximum at 450-480 nm. Flavoproteins (FAD) have absorption maxima at 430-500 nm and emission maxima at 520-590 nm. Elastin, a structural protein found in blood vessel walls (and in other connective tissues) shows emission of up to 500 nm when excited with UV light of up to 350 nm.

The inventors of the present application have found that by proper selection of an exciting wavelength range, a combination of a plurality of substances can be simultaneously detected (and visualized) in the tissue of interest, and such combined detection (visualization) of the simultaneous presence of two or more selected BASAs in said region provides accurate diagnosis of pathologic condition of the tissue, for example diabetic retinopathy (DR) identified from such combined imaging of retina.

More specifically, according to the invention, a given pathology condition can be detected or predicted in the biological tissue by imaging a predetermined region/location within the biological tissue via illumination of said region/location with an illumination pattern including the exciting wavelength range, which included a number N (N≥1) of exciting wavelength(s) capable of exciting two or more different BASAs to thereby induce and detect a combined emission response of said BASAs.

Preferably, the exciting wavelength range is relatively narrow, i.e. not exceeding 100 nm.

It should also be noted that the illumination pattern may include more than one exciting wavelength ranges, where at least one of the exciting wavelength ranges is intended to excite two or more different BASAs.

For example, when illuminating the retina with the exciting illumination in a range of about 350-380 nm (30 nm range width), the combination of NADH and flavins (soft exudates) and fatty acids (hard exudates) can be simultaneously visualized via their respective emission wavelengths in the combined response being detected, while being substantially simultaneously emitted in response to the exciting wavelength range, applied for example to the retina region of eye tissue. The simultaneous/concurrent presence of the combination of these substances in the retina region provides direct indication about existence/prediction of diabetic retinopathy (DR).

Another example of advantageous use of the technique of the invention is high-contrast visualization of blood vessels. More specifically, by using properly selected wavelengths of the illumination pattern, a combination of elastin and collagen within blood vessels can be visualized to enhance contrast of blood vessels. In this case, auto-fluorescence of these substances can be induced by illuminating blood vessels with the exciting wavelength range including 350 nm wavelength, both elastin and collagen have excitation at 350 nm and emission at 500 nm). When combining the exciting wavelengths for visualization of elastin and collagen (within blood vessel walls) with wavelengths dedicated to visualization of blood vessels (436 nm, 517 nm and 660 nm), the combined reflection from blood vessels and emission from blood vessel walls can be simultaneously detected to form a combined image with enhanced contrast of the imaged vasculature.

It should be understood that the above example of exciting wavelengths relates to visualization of the blood vessels, and any of these “visualization” wavelengths may be used in the illumination pattern in addition to one or more exciting wavelengths selected to cause the response of two or more substances which together are indicative of a specific pathological condition.

Thus, according to one broad aspect of the present invention, it provides a method for inspecting a biological tissue for one or more pathological conditions, the method comprising: illuminating a region within the biological tissue by an illumination pattern comprising a predetermined exciting wavelength range comprising a predetermined number N (N≥1) of predetermined exciting wavelengths selected to cause two or more auto-fluorescent responses of two or more predetermined different biological substances of types naturally existing in biological tissues; and detecting a combined spectral response of the biological tissue to said illumination pattern. The detected combined spectral response includes predetermined (expected) emission wavelengths of the auto-fluorescent responses of said two or more predetermined different biological substances (if present in the illuminated region) and may also include the exciting wavelength(s) returned (reflected) from structures/surfaces within the illuminated biological tissue. If it appears that the detected combined spectral response indeed includes the emissions of the respective substances in said region, this is indicative of a specific pathological condition.

Simultaneous detection of the combined spectral response of the illuminated tissue region including emission wavelengths of two or more different biological substances in response to a predetermined exciting wavelength range may be performed using a color camera device configured for concurrently detecting the plurality of auto-fluorescent responses of different wavelengths, and possibly also the reflection of exciting wavelength(s). The color camera has a pixel matrix configured and operable to define multiple detection channels of different colors. Such multi-channel pixel matrix may be defined by a suitable spectral filter. The use of color camera enables to obtain image data indicative of different detected wavelengths of the combined response and corresponding location data in the biological tissue where these different detected wavelengths are originated. It should also be understood that this provides higher signal to noise ratio of the detected fluorescent response over ambient lighting and reflection from the surrounding biological tissue.

Thus, the technique of the present invention is based on providing and utilizing assignment data, in which each pathological condition is assigned with a respective set of two or more different BASAs to be detected in a respective location/region within the biological tissue and corresponding imaging mode data defining one or more imaging modes to be performed on the respective location/region within the biological tissue. The imaging mode is characterized by a respective illumination pattern comprising a predetermined wavelength range (e.g. including one or more specific exciting wavelength(s)) selected to be capable of exciting two or more different BASAs causing their auto-fluorescent responses to be detected.

As indicated above, the wavelength range is preferably spectrally narrow such that its spectral width substantially not exceeds 100 nm. It should be understood that the “exciting wavelength” may be constituted by a central wavelength of a spectral profile (e.g., Gaussian profile).

Generally, the exciting wavelength(s) may be within a near UV spectrum causing auto-fluorescence emission from various BASAs in blue-green or red wavelength ranges, deep blue excitation with emission in green-red, green excitation with red or NIR emission, but not limited to it.

The illumination pattern may be in the form of pulses, e.g. including multiple excitation wavelengths.

The exciting wavelength range may include two or more discrete wavelengths (preferably spectrally close to one another) corresponding to exciting wavelengths of two or more different biological substances, which together define a selected combination of biological substances corresponding to a certain biological tissue condition to be inspected. In another example, the exciting wavelength range includes a single exciting wavelength capable of exciting two or more different biological substances. In yet another example, the illumination pattern includes two or more exciting wavelength ranges, where at least one of these exciting wavelength ranges includes exciting wavelength(s) for exciting multiple different biological substances. Common for all the embodiments of the illumination pattern with at least one exciting wavelength range is that it causes concurrent excitation of multiple (two or more) different biological substances, and enables simultaneous detection of combined emission response to identify the combination of the biological substances concurrently existing in the specific tissue region, which is indicative of a respective selected pathology condition.

The selected illumination pattern may include exciting wavelength(s) within a near ultraviolet (UVA) range (i.e. deep blue and UV range). This may include one or more exciting wavelengths (e.g. within the range of 340 nm to 420 nm) to cause excitation of a plurality of BASAs (plurality of different fluorophores); or may include a set of discrete exciting wavelengths or wavelength ranges having central wavelengths of 360 nm, 385 nm, 405 nm and 420 nm.

Expected auto-fluorescent responses of most of the biological substances, various combinations of which are of interest (i.e. are to be detected), are generally within the blue to green range of the visible spectrum. This includes various combinations of two or more of the following biological substances: human serum albumin, fatty acids (constituting lipid parts), NADH, flavoproteins (FAD), collagen, Elastin, amyloid, and AGEs (advanced glycation end products). Detection of presence in the tissue (typically eye tissue) of various combinations of any two or more of these substances provide indications to several pathologies.

As indicated above, the present technique utilizes detection of auto-fluorescence of naturally occurring biological auto-fluorescent spectral agents (BASAs). The technique is based on illuminating the tissue of interest in accordance with the absorption spectrum of the BASAs and filtering collected light in accordance with the emission spectrum. Further, as described in more detail below, the technique may utilize processing of the collected emission data to further increase the ability to differentiate between fluorescence emission and reflection of ambient light from the surrounding tissue.

The present technique thus omits the need for injecting contrast agents or other fluorescent agents to the patient. This shortens the time required for any medical testing and reduces possible negative reactions to the test (e.g. allergic reactions). Technically, this makes the present technique completely non-invasive as it does not require any injection prior to examination.

The ability to identify naturally occurring BASAs in selected body locations may provide indication for various pathologies. More specifically, the present technique enables detection of presence of BASAs, and accumulation of such materials in body regions where they do not normally occur, as well as pathological accumulation of such material in their natural environment. For example, detection of accumulation of a combination of two or more specific BASAs in the retina of a patient may indicate various damage to the retina or blood vessels therein.

The imaging mode may also be characterized by focal conditions of application of the predetermined illumination pattern to a region of the biological tissue. It should be understood that different wavelengths of illumination have different penetration depths in the tissue. Each spectral light component of the illumination pattern, while propagating through the tissue region being illuminated, induces auto-fluorescence from respective BASA(s), if such exist, in different layers of the tissue region along the illumination propagation path. The so-excited autofluorescence is scattered from the different layers while having its maximal intensity in the response of the layer located in a focal plane of the illumination (for the respective spectral light component).

Therefore, upon detecting such a response pattern (intensity variation) for at least one specific spectral component, the corresponding optimal focal length of the illumination can be identified for detection of specific BASA(s). Based on this data, a next imaging session may be performed on the same tissue region while controllably varying the focal length around said optimal focal length. This enables to accurately detect existence (and preferably also spatial distribution) of one or more specific BASAs in the tissue region.

Thus, according to another broad aspect of the invention, there is provided a method for inspecting a biological tissue, the method comprising: illuminating a region within the biological tissue by an illumination pattern comprising a predetermined exciting wavelength range comprising a predetermined number N (N≥1) of predetermined exciting wavelengths selected to penetrate through said region of the biological tissue and interact with different layers of the biological tissue in said region along a propagation path of the illumination pattern in said region to thereby cause auto-fluorescent response of one or more biological substances of types naturally existing in biological tissues; detecting the auto-fluorescent response during said propagation of the illumination pattern; analyzing data indicative of the detected auto-fluorescent response to identify spectral intensity variation of emission of one or more biological substances present in said region of the biological tissue along said propagation path and determine optimal focal length of the illumination pattern with respect to excitation of said one or more biological substances; and generating corresponding operational data comprising focal data for use in further measurements to optimize detection of existence and spatial distribution of said one or more predetermined biological substances in the tissue region.

Also, in some cases, the present technique can allow visualization of BASAs in their natural environment. For example, as described above, auto-fluorescence of elastin and collagen inside blood vessel walls can enhance contrast of blood vessels, thus improving the resolution with which angiography can be performed. The present technique can thus provide means of visualization of tissue damage processes in the retina, choroid and anterior chamber of the eye tissue, as well as improve the quality of visualization of blood vessels. For example, processes associated with monitoring ischemia, oxidative stress and/or proliferation of blood vessels. Accordingly, the simplicity of the testing according to the present technique provides a screening and diagnostic tool that can be used for simple monitoring of ongoing diseases and provide early detection.

According to another broad aspect of the invention, it provides a system for use in inspecting a biological tissue. The system comprises:

an imaging device configured and operable to perform two or more predetermined imaging modes and generate image data corresponding to each respective imaging mode, each imaging mode comprising at least one imaging session comprising: illumination of a selected location within a region of interest in the biological tissue by an illumination pattern comprising an exciting wavelength range selected to cause concurrent excitations and auto-fluorescent responses of two or more different biological substances of types naturally existing in biological tissues; detection of a combined spectral response of said location to the illumination pattern, and generation of corresponding image data indicative of the detected combined spectral response; and

a control unit comprising an imaging mode controller operating the imaging device to perform a selected one of the imaging modes, and a data processor analyzing the image data and generating output data indicative of the biological tissue condition, wherein said imaging mode controller is configured and operable in accordance with predetermined assignment data indicative of an assignment between each of said imaging modes and at least one corresponding biological tissue condition to be inspected, said imaging mode controller being responsive to user input about the at least one biological tissue condition of user's interest to select, based on the assignment data, respective imaging mode data and generate corresponding operational data to operate the imaging device to perform the at least one imaging session of the respective imaging mode.

As described above, in some embodiments, the exciting wavelength range has a spectral width of 100 nm or less.

The illumination pattern of each of the imaging modes comprises the exciting wavelength range which contains at least one exciting wavelength selected to excite and cause the auto-fluorescent responses of the different biological substances including two or more biological auto-fluorescent spectral agents (BASAs), whose concurrent presence in the selected location in the biological tissue presents a direct indication of a certain pathological condition of the biological tissue.

The imaging device comprises a light source unit configured and operable by operational data generated by the imaging mode controller to produce said illumination pattern corresponding to the selected imaging mode. The imaging device also includes focusing and imaging arrangements including inter alia an image detector configured and operable to collect the combined spectral response from the selected location.

The operational data with respect to the illumination pattern to be produced may also include focal data for operation of the focusing arrangement. Such focal data comprises data indicative of variation (sweeping) of focal condition of the illumination pattern around a predetermined optimal focal condition.

The image detector preferably includes a color camera device configured for concurrently detecting the plurality of auto-fluorescent responses of different wavelengths. The color camera may include a pixel matrix configured and operable to define multiple detection channels of different colors, such that the image data is indicative of different detected wavelengths of said combined response and corresponding location data in the biological tissue where said different detected wavelengths are originated. The color camera may be configured with collection channels of primary colors.

The data processor is configured and operable to receive and process the image data and identify, in said image data, image data pieces corresponding to the different emission wavelengths contained in said detected combined response, and determine a relation between said image data pieces to identify the corresponding location data in the biological tissue where said different emission wavelengths are originated.

The illumination pattern may include one or more illumination pulses of light comprising the selected exciting wavelength range.

The illumination mode data may include data about the illumination pattern assigned to the pathological condition, identifiable by the presence in the predetermined location of the two or more BASAs comprising two or more of the following: albumin, elastin, collagen, lipofuscin, fatty acids, LDL, NADH, flavins, porphyrins, amyloid and AGEs.

The detected combined spectral response may include the auto-fluorescent responses of the different biological substances excited by the exciting wavelength range, and reflection of one or more of exciting wavelengths of the illumination pattern from structures in the biological tissue being illuminated.

According to yet further broad aspect of the invention, there is provided a method for use in inspection of biological tissue. The method comprises:

providing predetermined assignment data comprising a plurality of k pathological conditions PC₁ . . . PC_{K}, each i-th pathological condition PCᵢ of said k pathological conditions being assigned with a respective at least one i-th imaging mode data defining imaging to be performed on at least one respective location within the biological tissue and being characterized by at least one respective i-th illumination pattern, each comprising a corresponding i-th exciting wavelength range which is selected to excite auto-fluorescent responses of a predetermined set of two or more different biological auto-fluorescent spectral agents (BASAs) whose concurrent presence in the selected location in the biological tissue presents a direct indication of said i-th pathological condition PCᵢ of the biological tissue;

in response to user input about the i-th pathological condition to be inspected for in the biological tissue, selecting, in said assignment data, the respective i-th imaging mode data, and generating corresponding operational data to an imaging device to implement said imaging mode in at least one imaging session on said predetermined location and detect a combined spectral response of said location to the illumination pattern, and generate corresponding image data indicative of the detected combined spectral response; and

processing the image data and providing output data about said i-th pathological condition.

### BRIEF DESCRIPTION OF THE DRAWINGS

In order to better understand the subject matter that is disclosed herein and to exemplify how it may be carried out in practice, embodiments will now be described, by way of non-limiting examples only, with reference to the accompanying drawings, in which:

FIG. 1 schematically illustrates a system for fluorescence imaging of biological tissue according to some embodiments of the invention;

FIG. 2 shows the excitation and emission spectra of bovine serum albumin;

FIG. 3 illustrates a method for use in inspection of biological tissue according to some embodiments of the invention;

FIG. 4 exemplifies operation of a technique for detection of biological auto-fluorescent agents (BASAs) according to some embodiments of the present technique;

FIGS. 5A and 5B show measured auto-fluorescence emission collected from a plasma drop using the preset technique, FIG. 5A shows raw representation of color image data and FIG. 5B shows summation of green and blue channels of the color image of FIG. 5A;

FIG. 6 is a picture of a serum drop on biological tissue, the drop is bright due to auto-fluorescent emission detected using some embodiments of the present technique; and

FIGS. 7A to 7C show image of serum drop placed on Petri dish, FIG. 7A shows raw conversion of the color image; FIG. 7B shows the image after processing by determining the relation between blue and green channels and FIG. 7C is a cross section of the image shown in FIG. 7B.

### DETAILED DESCRIPTION OF EMBODIMENTS

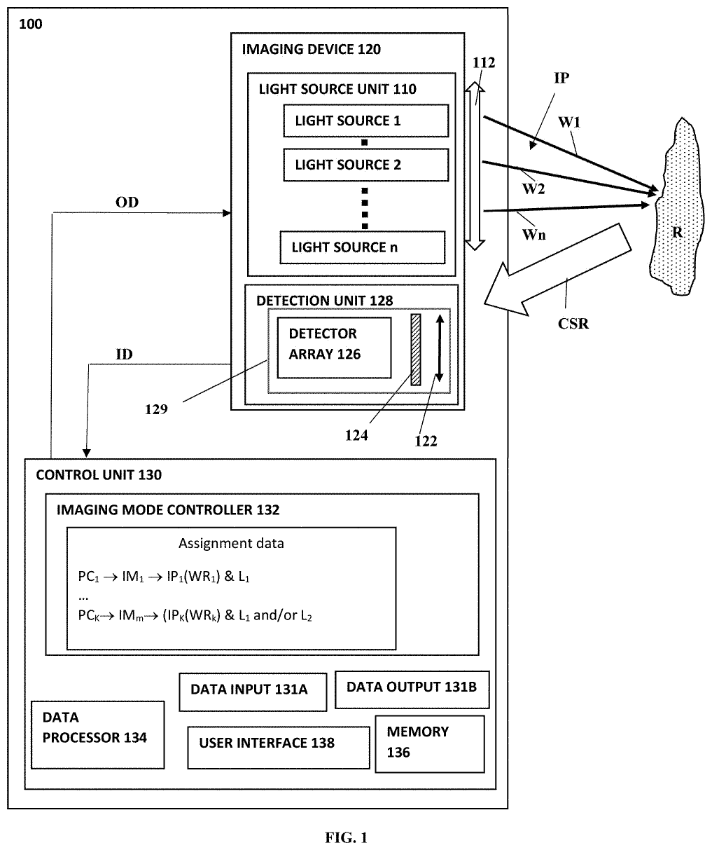

Reference is made to FIG. 1 schematically illustrating, by way of a block diagram, a system 100 of the present invention configured and operable for inspecting biological tissues by inducing and detection of auto-fluorescent responses of substances in a biological tissue R to detect presence or predict development of various pathology conditions in the biological tissue. More specifically, the system 100 is configured for inspecting the eye tissues, while the principles of the invention are generally not limited to this specific implementation.

The system 100 includes an imaging device 120 and a control unit 130. The imaging device 120 is configured and operable to perform a plurality of M (two or more) predetermined imaging modes IM₁ . . . IMₘ (defined by a plurality of K pathological conditions PC₁ . . . PCₖ to be detected/predicted), and generate corresponding image data for each imaging mode. The imaging mode comprises at least one imaging session, including illumination of a selected location/region in the tissue R by an illumination pattern IP comprising at least one predetermined (e.g. spectrally narrow) exciting wavelength range selected to cause concurrent excitation of multiple (two or more) predetermined different BASAs in the selected location/region, detection of a combined spectral response CSR of the illuminated/excited location including excitation-induced emission of the predetermined BASAs in said location; and generation of image data ID indicative of the detected combined spectral response.

Generally, the exciting wavelength range includes a number N (N≥1) of exciting wavelength(s). In the present not-limiting example of FIG. 1, such illumination pattern IP is exemplified by exciting wavelength range including multiple exciting wavelengths W₁ . . . Wₙ. It should, however, be understood that the invention is not limited to such multiple exciting wavelengths configurations. As described above, the case may be such that the exciting wavelength range actually includes a single relevant exciting wavelength, which, however, is selected to be capable of exciting multiple different predetermined biological substances, whose presence can be identified via detection of their emission responses in the detected combined spectral response.

It should be understood that thus, the principles of the invention are not limited to any specific configuration of excitation pattern, i.e. any specific number of exciting wavelengths or exciting wavelength ranges, provided the excitation pattern is selected to be capable of substantially concurrently exciting two or more different auto-fluorescent biological substances. Optionally, but in some embodiments preferably, the exciting wavelength range selected to excite multiple substances, is spectrally narrow (not larger than 100 nm).

Thus, the selected exciting wavelength range includes one or more predetermined exciting wavelength(s) selected for concurrently inducing auto-fluorescence responses of predetermined two or more naturally existing biological substances, the concurrent existence of which in the excited location in the biological tissue is indicative of the predetermined pathology condition of said tissue.

The control unit 130 is generally a computer system comprising such main functional utilities as data input and output utilities 131A, 131B, memory 136, and data processor 134. According to the invention, the control unit 130 includes an imaging mode controller 132 configured and operable to control the operation of the imaging device 120 to perform a selected one of the imaging modes in accordance with user input, entered via user interface 138, being indicative of user selection of the specific i-th pathological condition PCᵢ to be detected/predicted. The imaging mode controller 132 is thus response to the selected pathology condition relating data to utilize the assignment data and generate operational data OD to the imaging device (to its illumination unit).

The imaging mode controller 132 is configured and operable in accordance with predetermined assignment data indicative of an assignment between each of said imaging modes IM₁ . . . IMₘ and at least one corresponding biological tissue condition (pathological condition), where each imaging mode is characterized by a specific exciting wavelength range (including one or more selected exciting wavelengths) to be included in the illumination pattern applied to a predetermined location. In other words, according to the assignment data, the detection of i-th pathological condition PCᵢ is assigned with j-th imaging mode data IMᵢ characterized by g-th exciting wavelength range WR_{g} and a location in the tissue to be excited, where the exciting wavelength range WR_{g} includes exciting wavelength(s) corresponding to excitation of a predetermined set/combination of two or more auto-fluorescent substances, whose concurrent presence in the specific location provides direct indication of said pathology condition PCᵢ.

The assignment data may typically be pre-stored in the memory 136, and accessed by the imaging mode controller 132 in response to user input. The imaging mode controller 132 is thus configured for communication with the imaging device 120 to provide operational data OD thereto, in accordance with the selected assignment data defined by the user input.

As exemplified for simplicity in FIG. 1, pathology condition PC₁ is associated with the assigned image mode data IM₁ including illumination pattern data IP₁ characterized by an exciting wavelength range WR₁ (e.g. containing wavelengths W₁ and W₃ from the wavelengths/light sources for which the illumination unit is configured) and a location data L₁ (including focal data about focal location/region being imaged), . . . , and pathology conditions PCₖ is associated with the assigned image mode data IMₘ including illumination pattern data IPₖ defining an exciting wavelength range WRₖ (e.g. containing exciting wavelengths W₂ and W₃ and W₄) and a location data L₁ and possibly also additional location data L₂.

Thus, user provides input about the pathology condition of interest (via user interface 138), the imaging mode controller 132 operates to utilize the assignment data (e.g. stored in the memory 136, as the case may be) to select the corresponding imaging mode (assigned to detection of said pathology condition), and generates operation data OD to the imaging device 120. Image data ID resulting from the performance of the imaging mode is received at the data processor 134 which is configured and operable to analyze the image data ID and generate output data indicative of the biological tissue condition.

The imaging device 120 includes a light source unit 110 and a detection unit 128. The light source unit 120 is configured to generate multiple wavelengths W₁ . . . Wₙ and is controllably operable to selectively generate the required exciting wavelength range to create a selected corresponding illumination pattern to illuminate a selected location/region in the tissue. The detection unit 128 is configured and operable for collecting a combined spectral response of the tissue to said illumination pattern and generating corresponding image data ID.

Also provided in the imaging device is a focusing arrangement 112, enabling imaging of the specific region with required focal conditions with respect to a specific region/location and detection of the combined spectral response from said region/location. The operational data may include focal data corresponding to the required focal conditions.

In some examples, the focal data may include a predetermined location in the tissue on which the illumination pattern (having certain spectral characteristics) is to be focused (i.e. optimal focus of the system). In some other examples, the focal data may be indicative of a need to perform an imaging session with certain focus swept around the previously determined optimal location of a focal plane, with respect to one or more substances to be examined for their existence and spatial distribution in the biological tissue.

In the present not-limiting example, the light source unit 110 is illustrated for simplicity as including N light sources (e.g. LEDs), light source 1, light source 2, . . . light source n, capable of emitting, respectively N different wavelengths of wavelength ranges. The operational data OD provided by the imaging mode controller 132 includes activation data for a selected one or more of the light sources to generate at least one required exciting wavelength range (e.g. spectrally narrow) to form the illumination pattern corresponding to excitation of a corresponding combination of two or more substances in the certain tissue location.

The illumination pattern may include selected one or more wavelengths, and may be in the form of one or more illumination pulses, or a pulse train including a sequence of illumination of different wavelengths. Typically, temporal length of the illumination pattern may be within, or shorter, with respect to exposure time used by the detection unit 128.

The detection unit 128 includes a camera device 129 including a detector array 126 and including or being associated with an imaging lens arrangement 122 configured for imaging the region of interest R (or a specific location thereon) on the detector array 126. The detector array 128 is associated with a spectral filter 124 for filtering light being collected to detect the predetermined set of two or more emitted wavelengths in accordance with the imaging mode. The spectral filter may be a uniform filter allowing transmission of only selected wavelength range, or it may be a mosaic filter, such as Bayer filter.

For the purposes of the present invention, the camera device 129 is configured and operable as color camera or color detector array. Considering color detector arrays typically used in conventional cameras, they include three different types of detector cells configured for collection of light in different colors, typically primary colors of red, green and blue (RGB) spectra. The color camera device 129 (detector array and filter) used in the present invention provides a pixel matrix which defines multiple detection channels of different colors (spectral channels). As a result, the image data is indicative of different detected wavelengths of the combined response being detected and corresponding location data where said different detected wavelengths are originated. It should be noted that the wavelengths that are to be concurrently collected by the camera include emission wavelengths originated in the excited location in response to the exciting wavelength(s), if and when any of the responding substances exists in said location, and may also include one or more of exciting wavelengths being reflected from various elements/interfaces within the illuminated region and its surroundings.

For example, the camera device 129 may be configured as described in WO 2020/183462, which is assigned to the assignee of the present application and is incorporated herein by reference with respect to a specific example of the color camera configuration. In such camera device, a detector array includes a plurality of detector cells, including detector cells of two or more different types arranged in a predetermined array (two-dimensional array) and differing from one another in their spectral response functions, i.e. the sensitivity of detector cells to light of different wavelengths. Thus, output image data collected by one type of detector cells provides an image of the field of view using a certain wavelength range (corresponding to the spectral response of the detector cells). The detector cells of different types are properly arranged (e.g. in an interlaced order) within a common plane of the detector array, such that images collected simultaneously by each of the different types of detector cells that are associated with a common field of view, thereby not requiring additional registration processing.

In some embodiments, the detection unit 128 may further include an additional spectral filter, for example configured to prevent collection of short wavelengths, e.g. near UV or wavelengths below 450 nm.

Generally, the system 100 of the present invention may be configured to provide illumination pattern that includes selected exciting wavelength(s) to excite auto-fluorescent responses of predetermined two or more different biological auto-fluorescent spectral agents (BASAs). Such BASAs include biological materials/substances, such as albumin, elastin, collagen, lipofuscin, fatty acids, LDL, NADH, flavins, porphyrins, amyloid and advanced glycation end products (AGEs). Additional substances that can be classified as BASAs are known and may also be considered to be detected in various combinations for the purpose of the present technique in accordance with pathological conditions to be detected in various biological tissues to be inspected.

Generally, each biological auto-fluorescent substance is characterized by its exciting spectral range and a corresponding fluorescent spectral response. According to the invention, in order to identify existence/prediction of a specific pathology condition of a biological tissue, a set of two or more BASAs is defined as being unusual/abnormal for concurrent appearance (possible in certain unusual amount) in a specific location. Further, for all of the BASAs of the selected combination/set, at least one exciting wavelength range (in some embodiment being spectrally narrow, i.e. having spectral bandwidth substantially not exceeding 100 nm) is selected including one or more exciting wavelengths suitable to concurrently excite the predetermined set of the BASAs. This enables fast (single imaging session/single exposure) and accurate detection of the existence or absence of the entire combination of BASAs in the selected location providing direct indication to the corresponding pathological condition(s) of the tissue, assisting in diagnosis of selected pathologies. This may enable a physician to determine patients' health condition and identify early stages of various conditions. Alternatively, this may enable screening for conditions that are yet to be developed in patients at risk for developing such conditions.

For example, albumin, or Human serum Albumin is the most abundant protein in the human blood plasma. Albumin constitutes about half of serum protein. It is produced in the liver; it is soluble in water and is monomeric. Among Albumin's biological functions are transporting of hormones, fatty acids, and other compounds. Albumin also assists in pH buffering and maintaining oncotic pressure within blood vessels. Human plasma albumin has an excitation peak at about 280 nm and emission maximum at 330-350 nm. Bovine serum excitation in the range 340-400 nm results in emission at 450-550 nm. Human plasma has an excitation maximum at 400-420 nm nm and emission maximum at 460-520 nm. Typically, detection of high depositions of albumin outside the blood stream may indicate leakage from blood vessels, which may be associated with various known pathologies.

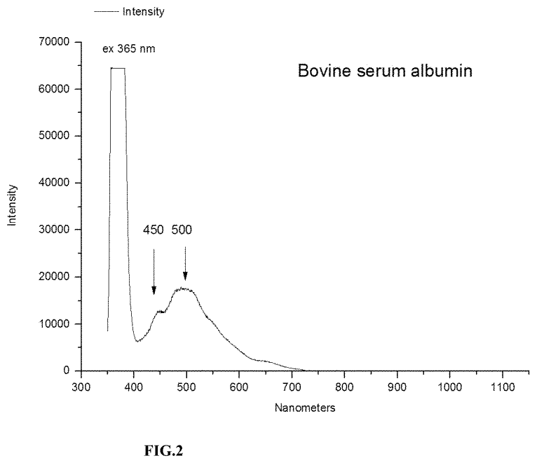

In this regard, reference is made to FIG. 2 exemplifying measured absorption and fluorescent emission spectrum of bovine serum albumin, having very close properties as Human serum albumin. As shown, albumin absorbs light at excitation range between 350 nm and 400 nm and responds by emission of light peaking at 450-550 nm. The 450 nm shoulder peak and 550 nm peak are marked in FIG. 2.

Lipofuscin is a dominant macular fluorophore that absorbs blue light with a peak excitation wavelength of 488 nm and emits red light at a peak wavelength of 630 nm. Lipofuscin is found in the Retinal pigment epithelium (RPE) and is a heterogeneous mixture that derives its auto fluorescent properties from bisretinoid compounds, which are metabolic byproducts of vitamin A and the visual cycle. Bisretinoids are initially formed in photoreceptor outer segments and then deposited in the RPE as lipofuscin, accumulating in RPE lysosomes with age. Lipofuscin also increases in degenerative disorders, including Age-related Macular Degeneration (AMD), and macular dystrophies such as Best and Stargardt disease. The distribution of lipofuscin, and consequently the distribution of its auto-fluorescence, is greatest in the posterior pole, however limited in the fovea, and decreases towards the periphery.

Advanced glycation end products (AGEs) are products of non-enzymatic binding of sugars to proteins, lipids and more. Such end products may be generated due to high levels of blood sugar. AGEs are involved in the pathogenesis of diabetic retinopathy (DR) as a result of damage to blood vessel formation, proliferation and architecture. Furthermore, AGEs cause tissue damage through activation of inflammatory processes, induction of apoptosis (programmed cell death) and more. Accumulation of AGEs may lead to both interrupted vessel metabolism and cellular metabolism, with leakages from both blood vessels and cells. AGEs can form sediments all over the retina, forming cross-links and/or accumulate in the vicinity of damaged blood vessels with leaking plasma, where AGEs can also be found.

The technique of the present invention provides for concurrent detection of the existence of such pathological condition as DR by direct detection (and visualization) of the combination of both AGEs and plasma in a retina region/location of the eye tissue. To this end, the imaging mode is performed on the retina using the illumination patterns including the exciting wavelength range of 350-400 nm including the AGEs excitation wavelength of 385 nm and plasma excitation wavelength of 400 nm. If both the AGEs and plasma indeed concurrently exist in the retina region, the combined spectral response of the illuminated region includes an emission spectrum including the AGEs emission wavelength of 440 nm and the plasma emission wavelength of 490 nm (e.g. combined emission spectrum of a 430-450 nm). Such detection of the concurrent existence of AGEs and plasma in the retina region provides direct detection/prediction (early diagnostics) of the DR condition in a patient. In this regard, it should be noted that, due to the fact that plasma can leak in different pathologies, detection/visualization of plasma only does not assist in DR diagnosis, while detection of concurrent existence of the plasma together with AGEs which sediment mostly in diabetes, directly indicates towards the DR pathological condition.

Another example of the advantageous effect of the technique of the present invention for early detection of DR related condition may be by concurrent detection of the presence of both NADH and flavoproteins in soft exudates (among the retinal signs of DR). The combination of both NADH and flavoproteins can be detected when illuminating the retinal region with the illumination pattern including exciting spectral range of 360-420 nm including NADH excitation wavelength of 365 nm and Flavoproteins excitation wavelength of 430 nm. Image data indicative of the detection of the combined spectral response including NADH and Flavoproteins emissions corresponds to predicted DR condition. Differentiation between both substances is based on emission wavelengths: NADH responds to the above excitation by emission wavelength of about 465 nm (generally, emits within the range of 450-480 nm (blue)) and flavoproteins responds by emission of 550 nm (generally, emits within the range of 520-590 nm (green)).

It has been previously found that detection of flavoproteins in the retina can serve as a predictor of DR development in diabetic patients that are yet to develop DR. Combining the concurrent detection of the existence of both NADH and flavoproteins by applying the same narrow range of excitation, allows early detection with higher accuracy (due to visualization with enhanced contrast) of the existence of the DR condition, and can thus provide improved screening for DR. Further, is indicated herein the use of color camera in the detection device provides detection channels of different wavelength ranges (colors) which enables separate detection of emission from NADH (blue channel) and flavoproteins (green channel).

According to another non-limiting example, the invention provides for detection of such pathological condition as age related macular degeneration (AMD), via the predetermined imaging mode performed in the retina region of the eye tissue, according to predetermined assignment data. The assignment data defines the combination of biological auto-fluorescent substances which, when exist together in the same region, provide indication of said condition. Such substances include, for example, Amyloid which can be excited by 350 nm wavelength to respond by 450 nm emission, and fatty acids excitable by 350 nm wavelength and responding by 475 nm emission.

Amyloid is commonly known as a defected protein that forms sediments in the brain and takes part in the pathogenesis of Alzheimer's disease. However, amyloid is also known to take part in the development of other diseases, such as amyloidosis. Furthermore, amyloid is known to form sediments all over the retina and can serve as indicator for AMD. Drusen exudates are a special type of exudates that can be found in AMD and contain a variety of substances, including many different proteins, lipids and others. The combination of both amyloid and fatty acids within Drusen exudates can be visualized using the technique of the invention by exciting the retina region with an illumination pattern including the exciting wavelength range containing the common exciting wavelength of 350 nm, and identifying, in the detected combined emission response, the emission wavelengths of 450 nm and 475 nm (blue range of the visible spectrum of 430-480 nm). Detection of simultaneous presence of amyloid and fatty acids within Drusen exudates in the retina can provide early and more accurate diagnosis of AMD.

Additionally, or alternatively to detection of simultaneous existence of various combinations of BASAs in the retinal region of the eye tissue indicative of pathological conditions of various types, one or more pathological conditions may be identified via detection of simultaneous presence of a variety of substances (BASAs) in other regions/locations, for example, the anterior chamber of the eye including the cornea and lens. For example, detection of simultaneous presence of AGEs and flavoproteins in the cornea region may provide indication for severity of DR (especially proliferative DR). The use of an illumination pattern including excitation wavelength range of 360-420 nm (including AGEs exciting wavelength of 385 nm and flavoproteins exciting wavelength of 430 nm), and detection spectra including a range of 430-450 nm (for detection of AGEs emission of 440 nm) and a range of 520-590 nm (for detection of flavoproteins emission of 550 nm), provides direct indication to the existence of these substances in the cornea. As indicated above, the use of color camera can provide separation in image data pieces between blue and green channels providing registration of AGEs and flavoproteins within a single imaging session.

Accordingly, detection of BASAs in the retina and anterior chamber (cornea and eye lens) of the eye can provide an indication associated with various medical pathologies, including but not limited to, diabetic retinopathy (DR), age-related macular degeneration (AMD), glaucoma and other retinal and choroidal diseases. Typically, such pathologies may produce ischemic, as well as metabolic changes in the retina, choroid and anterior chamber. Disruption of normal tissue metabolism due to such pathological processes will result in BASA leakages from blood vessels and/or tissue cells, forming sediments in the relevant region of interest. It should be noted that generally, BASAs present within blood vessels or within the tissue, exhibit non-detectable fluorescent response due to interference occurring from other auto-fluorescent materials in their vicinity (e.g. hemoglobin inside blood vessels). Thus, leaks from blood vessels or from tissue cells may be detected based on the presence of BASAs outside of their natural environment.

Additionally, in some embodiments, the present technique may utilize detection of a combination of BASAs, such as elastin and collagen, when found in normal amounts in normally functioning tissue. This technique may provide high contrast imaging of blood vessels, where such proteins are found in the blood vessel walls and increase imaging contrast by additional of auto-fluorescent response.

Turning back to FIG. 1, the system 100 may generally carry pre-stored assignment data indicative of a relation between pathological conditions to be identified and suitable imaging modes, where each imaging mode is defined by the respective at least one exciting wavelength range and respective expected combined radiation response corresponding to predetermined substances to be identified in the biological tissue and an optimal location/region in the biological tissue to be imaged. This enables to determine existence and may possibly also indicate amounts of selected BASAs as described above, in selected biological tissues.

Thus, the novel approach of the invention provides for fast and effective detection/prediction of various pathological conditions. This approach is based on the inventors' understanding of auto-fluorescent properties of various biological auto-fluorescent agents (BASAs) and the relation between various combinations of the BASAs and pathological conditions. Such relations enable to define one or more selected imaging modes per the pathological condition to be monitored. Different imaging modes associated with the detection of the same pathological condition may be different in the exciting wavelength ranges and/or locations/regions to be imaged. In accordance with the selected imaging mode, the illumination pattern includes exciting wavelength range(s) each formed of one or more discrete wavelengths exciting two or more different substances.

In some configurations, the detected response of the tissue includes also reflection(s) of the exciting wavelength(s), or the illumination pattern may also include non-exciting wavelengths selected to provide reflection response from the tissue, as the case may be. Detection of the tissue reflections enables certain visualization of the surrounding tissue. This may be used e.g. in visualization of blood vessels using auto-fluorescence of elastin and collagen combined with high contrast imaging of the blood vessels based on reflected illumination.

The data processor 134 is configured to receive image data ID from the imaging device 120 being indicative of the detected combined spectral response of the tissue region of interest, and process the image data to determine data indicative of the specific BASAs in the inspected tissue in correspondence with the imaging mode being used. Generally, as indicated above, the image data may include two or more image data pieces (generally three), or channels, each relating to collected light in different wavelength ranges contained in said detected combined response of the tissue. The data processor 134 thus operates for processing the spectral channels of the image data and determining a relation between selected spectral channels corresponding to relative locations of the responding substances within the tissue. More specifically, the data processor 134 may determine pixel-by-pixel relation, e.g. by determining for example a ratio between green and blue channels, sum of the channels, difference of the channels, etc. The data processor 134 may thus be configured for receiving the image data, extracting the different spectral channels and processing image data pieces in one or more, generally two or more, selected channels.

Generally, as indicated above the selected illumination pattern provides selection of exciting wavelength(s) for simultaneous detection and visualization of various combinations of BASA substances. For example, using specific exciting wavelengths in the range of 350 nm to 380 nm enables simultaneous detection (and visualization) of the combination of NADH, flavins and fatty acids. Typically, the NADH and flavins may be visualized in soft exudates and fatty acids are visualized in hard exudates.

Additionally, for example, selection of specific wavelengths around 360 nm enables simultaneous visualization of combination of both elastin and collagen within blood vessel walls. This may be used to provide enhanced contrast imaging of blood vessels. In that case, auto-fluorescence resulting from illumination patterns may be used in addition to imaging, using reflection of light from selected wavelength ranges and corresponding processing of the collected image data. For example, in such configuration the illumination pattern may further include illumination at 436 nm, 517 nm and/or 660 nm to provide improved contrast due to reflection and absorbance relations of light from blood vessels.

It should be noted that generally, auto-fluorescence of BASAs is relatively weak. However, the inventors of the present technique have identified that the use of illumination having one or more, but generally a few wavelengths, provides concurrent excitation of multiple different BASAs and corresponding concurrent multiple emissions that can be detected by a detector array of color camera enabling post processing of the output of the color channels of the detector array. This enables to enhance contrast of fluorescence emitted from the tissue and detect presence of the combination of BASAs, providing images of their distribution in a tissue. Such imaging is highly beneficial in relatively superficial tissues, or such that are easily visible, such as the retina, cornea and eye lens.

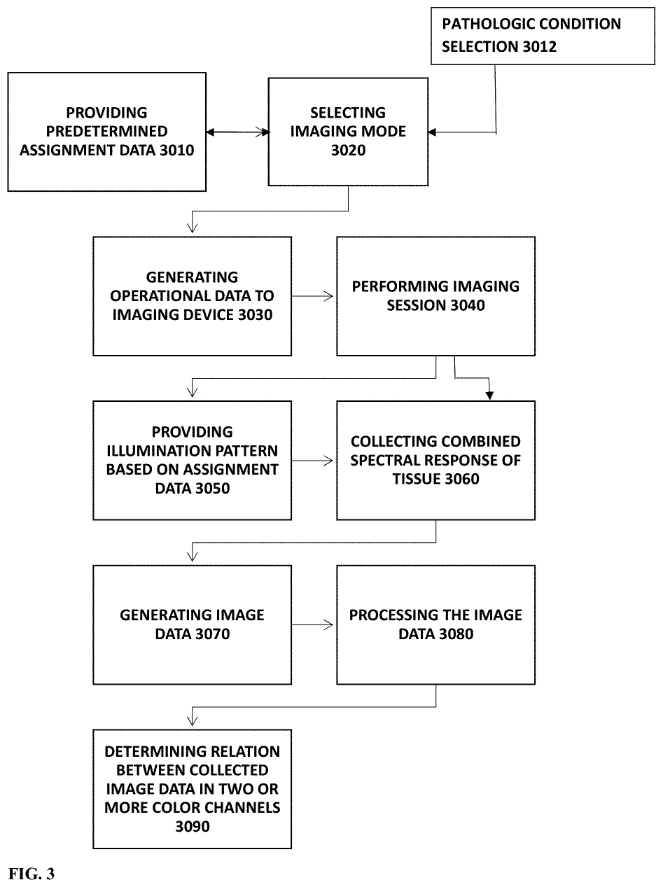

Reference is now made to FIG. 3 illustrating in a way of a flow diagram a method of the invention for use in inspection of biological tissue. As shown, the method includes providing predetermined assignment data (step 3010) indicative of a selected number of k pathological conditions PC₁ . . . PC_{K}, where each i-th pathological condition PCᵢ is assigned with respective imaging mode data defining imaging to be performed on a respective locations within the biological tissue by the use of a corresponding illumination pattern including the spectrally narrow exciting wavelength range, in accordance with corresponding pathological condition to be detected. In response to the user's selection of the pathological condition (step 3012), the assignment data is accessed and analyzed to select the respective imaging mode data (step 3020), and generate corresponding operational data OD to the imaging device (step 3030). The imaging device is activated by the image mode data to perform at least one corresponding imaging session (step 3040). The imaging session includes generation of the predetermined illumination pattern towards the predetermined location according to the assignment data (step 3050), collection/detection of the combined response (step 3060), and generation of image data ID (step 3070).

The so-produced image data may be directed for real-time processing, i.e. so-called on-line mode (step 3080), or may be stored to be accessed and processed later (off-line mode), for providing output data about the tissue status in relation to said i-th pathological condition of interest, whether the predetermined combination of selected BASAs exists in the selected location or not, and/or whether the amount of BASAs in such combination corresponds to the pathological condition or not. The processing may include analyzing the image data for one or more image data pieces associated with color channels of the collected image, and determining a relation between two or more color channels of the image data (step 3090) to determine data on existence of selected combination of BASAs in the selected location.

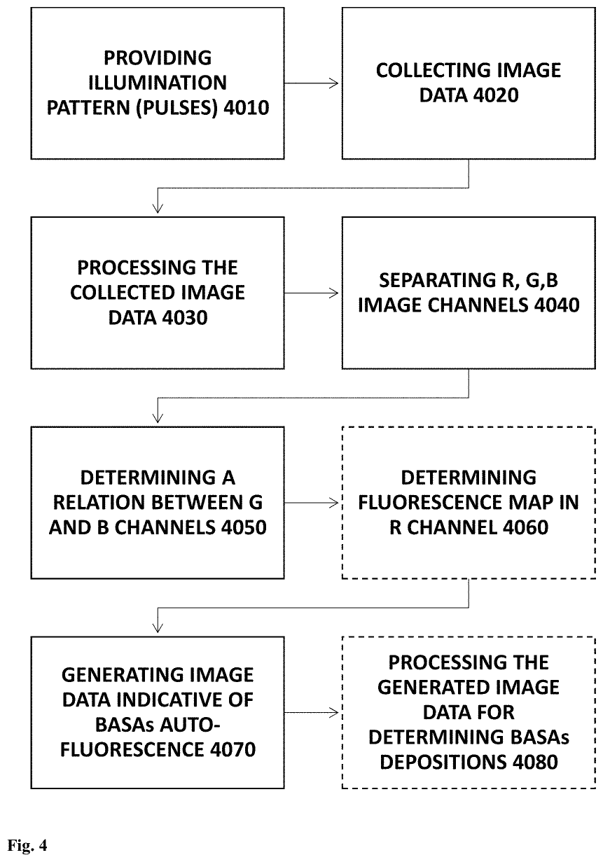

As indicated above, the present technique may utilize a color camera providing RGB type image data. Reference is made to FIG. 4 exemplifying the imaging and data processing stages according to some embodiments of the invention. As shown, the technique utilizes providing an illumination pattern (step 4010). The illumination pattern may generally include one or more pulses including the selected exciting wavelength range (e.g. a set of two or more exciting wavelengths) generated, for example by one or more LEDs or laser units.

The illumination pattern may include one or more pulses of a wavelength range, e.g. selected between 340 nm and 420 nm, to provide excitation of selected two or more BASAs. In some configurations, the illumination pattern may also include at least one additional selected exciting wavelength range including a second one or more wavelengths, e.g. selected between 460 nm and 540 nm. Such second wavelength range may be selected to provide excitation of additional BASA(s) such as Lipofuscin. In such configurations, the technique may utilize filtering of collection of auto-fluorescent emission corresponding to the second exciting wavelength range.

Generally, the present technique utilizes collecting image data 4020 from the inspected tissue with certain synchronization with illumination. In some configurations, the image data is collected using a color camera providing image data having different color channels (e.g. primary colors such as RGB). The color camera utilizes spectral filters differentiating the spectral channels to provide a color image representation of the tissue, and enabling certain spectral processing of the collected image, while obviating the need for any image registration processing that may be required in the case of the use of different detector arrays for collecting monochromatic image data and associated with corresponding different spectral filters. The detected image data is transmitted for processing (step 4030) for identifying auto-fluorescence emission of the predetermined BASAs and increasing signal to noise of the detected fluorescence. The processing generally includes separating the different spectral channels associated with Red, Green and Blue channels (step 4040), and determining a relation between the selected channels (step 4050), typically between the green and blue channels. The relation provides for pixel-by-pixel mapping of the selected channels, thereby enabling to determine variation between collected light in the selected channels. In configurations utilizing the second wavelength range (e.g. second set of excitation wavelengths), the processing may operate to determine a fluorescence map related to collected light in the red channel (step 4060) being generally indicative of fluorescence emission of Lipofuscin, having peak emission around 630 nm. Hence, in this example, the emission data is collected simultaneously from the B and R channels.

Using the fluorescence maps determined based on the different color channels in step 4050 and/or step 4060, the provided output data is (step 4070) indicative of auto-fluorescence distribution of the selected BASAs within the image detected by the detector array, enabling to identify existence of depositions of the selected BASAs in the tissue. In some configurations, the image data may be presented to an operator for analysis. In some additional configurations, the image data may be further processed (step 4080) for determining the BASAs depositions, and typically abnormal BASAs depositions as compared to normal typical material composition of the inspected region. As indicated above, various pathologies result in formation of depositions outside of blood vessels. Such depositions may be visible in the image data as bright spots due to BASAs auto-fluorescence detected by the present technique.

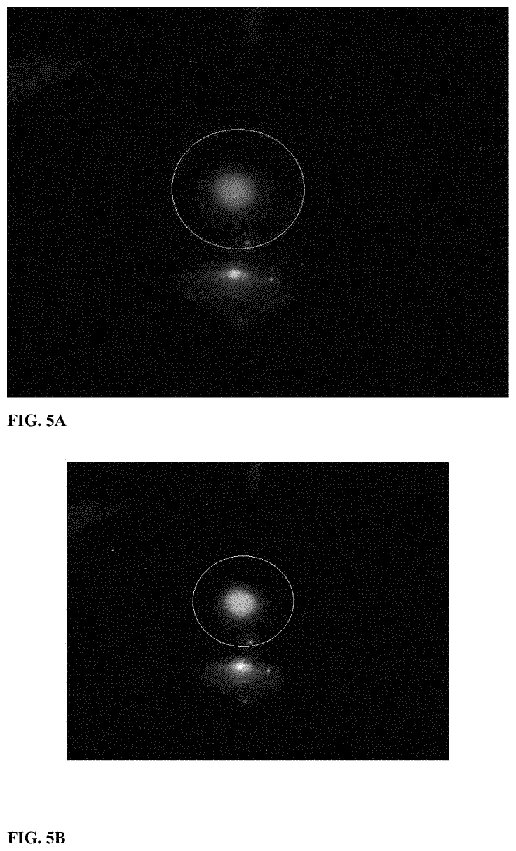

Reference is made to FIGS. 5A and 5B exemplifying auto-fluorescence emission detected from a plasma drop using the above-described technique of the present invention. FIG. 5A shows auto-fluorescence detected from a plasma drop collected using a color camera utilizing a band pass filter 450-550 (allowing transmission of light having wavelength in the range of 450-550 nm) represented as a raw image. FIG. 5B shows summation of green and blue channels of the color image collected in FIG. 5A. The drop of plasma was placed on a glass plate and put under excitation illumination of 365 nm. The auto-fluorescence emission is detected centered around 500 nm and is shown in FIGS. 5A and 5B in the circled bright spot. The lower spot is a result of undesired reflection.

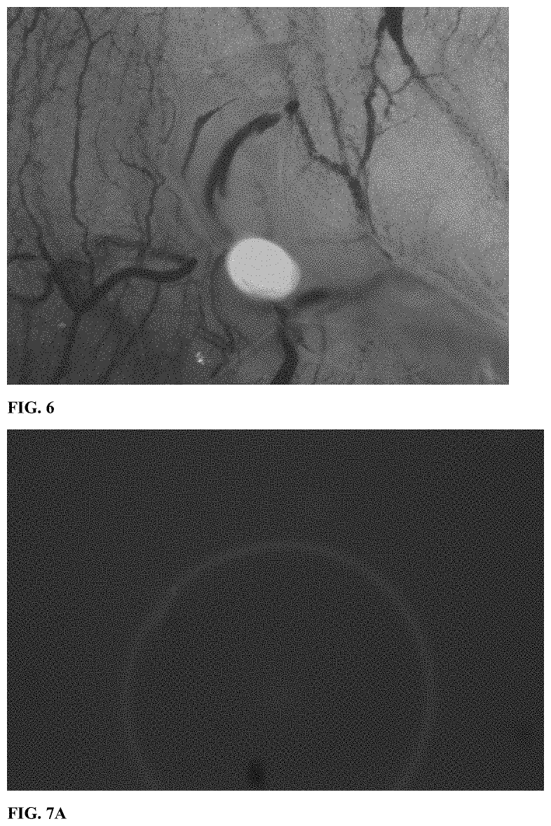

Reference is made to FIG. 6 showing experimental results of auto-fluorescence detected from a serum drop on biological tissue. The region was illuminated by pulses of an exciting wavelength range including an excitation wavelength of 360 nm, the combined spectral response was detected using a color camera with a band-pass filter transmitting light in the range 450 nm-550 nm. The drop shows brightness over the tissue background due to higher levels of BASAs, such as albumin in the plasma, over the general concentration in the normal biologic tissue.

As indicated above, the present technique may utilize selected filters to provide collection of auto-fluorescent emission over background reflection. Further, as indicated, in some embodiments of the present invention, color (e.g. RGB) camera may be used with the standard Bayer filer arrangement, and color channels in the collected image data may be processed to improve detection of auto-fluorescent emission.

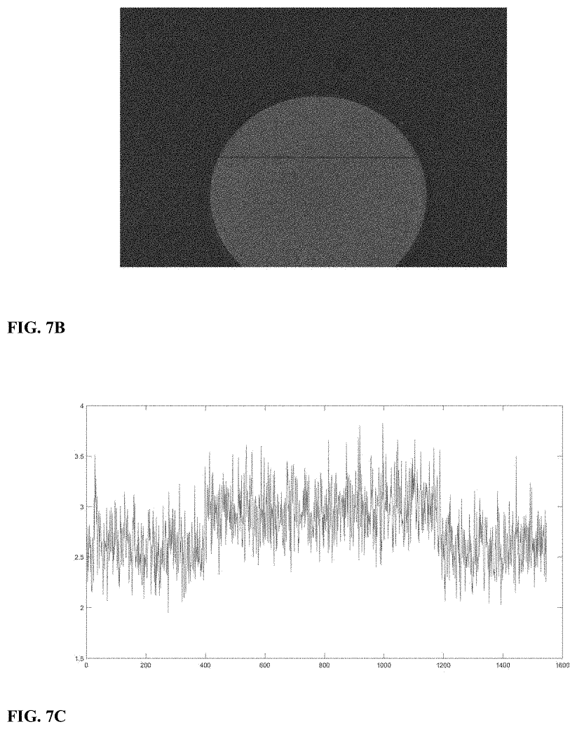

FIGS. 7A to 7C show image of serum drop placed on Petri dish. FIG. 7A shows a raw representation of the color image; FIG. 7B shows the image after processing for determining relation between blue and green channels; and FIG. 7C shows a plot along cross section of the image shown in FIG. 7B. The drop of plasma was illuminated by illumination pulsed pattern including an exciting wavelength having wavelength of 360 nm, and the color camera was used with an additional band pass filter transmitting light in the range of 450 nm-550 nm. As shows in FIG. 7A, the auto-fluorescent emission is visible along the circumference of the drop. However, additional processing of the image data (corresponding to the detected combined spectral response) and determining pixel-by-pixel relation between the green and blue channels provides improved indication of the auto-fluorescent emission as shown in FIG. 7B. FIG. 7C further illustrates the auto-fluorescent emission from the plasma drop over the background noise showing average emission of 3 (A.U.) over background average of 2.5 (A.U.) associated with reflection from the background and ambient light.

Accordingly, as described above, the technique of the present invention provides for detection of auto-fluorescent emission of various combinations of biological agents (BASAs), including fatty acids, albumin, elastin, collagen, lipofuscin, LDL, NADH, flavins, porphyrins, amyloid and advanced glycation end products (AGEs). The present technique enables a simple and robust detection technique providing an indication of various pathologies that may be associated with depositions of combinations of such BASAs in biological tissues, e.g. due to blood leaks of other pathological processes.

The present technique thus enables detection of presence of various BASAs including albumin and other naturally occurring fluorescent materials; this enables detection of pathologies that may be associated with leaks from blood vessels or other pathologies associated with aggregation of plasma, proteins, lipids, sugars, nucleic acids or other cellular or extracellular materials in tissues. The present technique may be subjected to regular standards for use in near-UV spectrum illumination while providing such illumination for generally very short pulses, for example below 1/60 second. It should be noted that the pulse duration is generally determined in accordance with imaging requirements, and pulse intensity. The technique requires no intrusive operations needed and does not need any administration of Fluorescein or other fluorescent dyes to patients' veins. Accordingly, the present technique may provide an immediately available test that does not require any preparations, as BASA auto-fluorescence in a physical effect of naturally occurring materials. The present technique may be highly advantageous for ocular imaging, providing high quality imaging, including sensitivity to material deposits within the retina, choroid and anterior chamber of the eye (cornea and eye lens).

1. A system for use in inspecting a biological tissue, the system comprising:
an imaging device configured and operable to perform two or more predetermined imaging modes and generate image data corresponding to each respective imaging mode, each imaging mode comprising at least one imaging session comprising: illumination of a selected location within a region of interest in the biological tissue by an illumination pattern comprising an exciting wavelength range selected to cause concurrent excitations of two or more different biological substances of types naturally existing in biological tissues to thereby concurrently induce auto-fluorescent responses of said two or more different biological substance; detection of a combined spectral response of said location to the illumination pattern, and generation of corresponding image data indicative of the detected combined spectral response; and
a control unit comprising an imaging mode controller operating the imaging device to perform a selected one of the imaging modes, and a data processor analyzing the image data and generating output data indicative of the biological tissue condition, wherein said imaging mode controller is configured and operable in accordance with predetermined assignment data indicative of an assignment between each of said imaging modes and at least one corresponding biological tissue condition to be inspected, said imaging mode controller being responsive to user input about the at least one biological tissue condition of user's interest to select, based on the assignment data, respective imaging mode data and generate corresponding operational data to operate the imaging device to perform the at least one imaging session of the respective imaging mode.

2. The system of claim 1, wherein said exciting wavelength range has a spectral width substantially not exceeding 100 nm.

3. The system of claim 1, wherein the illumination pattern of each of the imaging modes comprises the exciting wavelength range containing at least one exciting wavelength selected to excite and cause auto-fluorescent responses of the different biological substances including two or more biological auto-fluorescent spectral agents (BASAs), whose concurrent presence in the selected location in the biological tissue presents a direct indication of a certain pathological condition of the biological tissue.

4. The system of claim 1, wherein the imaging device comprises: a light source unit configured and operable by operational data generated by the imaging mode controller to produce said illumination pattern corresponding to the selected imaging mode; and an image detector configured and operable to collect the combined spectral response from the selected location.

5. The system of claim 1, wherein the imaging device comprises a focusing arrangement.

6. The system of claim 4, wherein said image detector comprising a color camera device configured for concurrently detecting the plurality of auto-fluorescent responses of different wavelengths.

7. The system of claim 6, wherein said color camera device has at least one of the following configurations: the color camera device comprises a pixel matrix configured and operable to define multiple detection channels of different colors, such that the image data is indicative of different detected wavelengths of said combined response and corresponding location data in the biological tissue where said different detected wavelengths are originated; and color camera is configured with collection channels of primary colors.

8. The system of claim 6, wherein said data processor is configured and operable to receive and process the image data by carrying out at least one of the following:
identifying, in said image data, image data pieces corresponding to the different emission wavelengths contained in said detected combined response, and determining a relation between said image data pieces to identify the corresponding location data in the biological tissue where said different emission wavelengths are originated; and
identifying spectral intensity variation of emission of one or more biological substances present in the tissue along a propagation path of the illumination pattern through the tissue, and determining an optimal focal length of the illumination pattern with respect to excitation of one or more of the biological substances; and generating corresponding operational data comprising focal data for use in further measurements to optimize detection of existence and spatial distribution of said one or more biological substances in the tissue region.

9. The system of claim 1, wherein the illumination pattern comprises one or more illumination pulses of light comprising the selected exciting wavelength range.

10. The system of claim 3, wherein the imaging mode comprises the illumination pattern assigned to the pathological condition identifiable by the presence in the predetermined location of the two or more BASAs comprising two or more of the following: albumin, elastin, collagen, lipofuscin, fatty acids, LDL, NADH, flavins, porphyrins, amyloid and AGEs.

11. The system according to claim 1, wherein the illumination pattern comprises at least one additional exciting wavelength range selected to excite at least one additional biological substance to cause at least one additional fluorescent response of said at least one additional biological substance to be included in said detected combined spectral response.

12. The system according to claim 1, wherein said combined spectral response comprises the auto-fluorescent responses of the different biological substances excited by the exciting wavelength range, and reflection of one or more of wavelengths of the illumination pattern from structures in the biological tissue being illuminated.

13. The system of claim 11, wherein the illumination pattern includes one or more visualization wavelengths selected to enhance imaging of blood vessels in vicinity of the location being imaged, such that the image data includes a combined image of the blood vessels and said two or more excited substances.

14. The system of claim 13, wherein said one or more visualization wavelengths includes one or more exciting wavelengths selected to excite substances associated with blood vessel structures.

15. The system of claim 14, wherein said one or more visualization wavelengths includes at least one of 436 nm, 517 nm and 660 nm wavelengths.

16. The system of claim 15, wherein the illumination pattern includes 350 nm wavelength for exciting elastin and collagen substances.

17. The system according to claim 1, wherein said assignment data comprises data indicative of pathological condition relating to diabetic retinopathy (DR) assigned with the one or more imaging mode to be performed on the location in a retina region utilizing comprising at least one of the following:
(a) the illumination pattern comprising the exciting wavelength range comprising 385 nm and 400 nm wavelengths selected to induce simultaneous auto-fluorescence response of AGEs and plasma substances,
(b) the illumination pattern comprising the exciting wavelength range comprising 365 nm and 430 nm wavelengths selected to induce simultaneous auto-fluorescence response of NADH and flavoproteins substances.

18. The system according to claim 1, wherein said assignment data comprises data indicative of pathological condition relating to at least one of the following:
macular degeneration (AMD) assigned with the one or more imaging mode to be performed on the location in a retina region comprising the exciting wavelength range including 350 nm wavelength selected to induce simultaneous auto-fluorescence responses of amyloid and fatty acids substances; and
proliferative diabetic retinopathy (DR) assigned with the one or more imaging mode to be performed on the location in a cornea region comprising the exciting wavelength range including 385 nm and 430 nm wavelengths selected to induce simultaneous auto-fluorescence responses of AGE and flavoproteins substances.

19. A method for use in inspection of biological tissue, the method comprising:
providing predetermined assignment data comprising a plurality of k pathological conditions PC₁ . . . PC_{K}, each i-th pathological condition PCᵢ of said k pathological conditions being assigned with a respective at least one i-th imaging mode data defining imaging to be performed on at least one respective location within the biological tissue and being characterized by at least one respective i-th illumination pattern, each comprising a corresponding i-th exciting wavelength range which is selected to induce substantially simultaneous auto-fluorescent responses of a predetermined set of two or more different biological auto-fluorescent spectral agents (BASAs) whose concurrent presence in the selected location in the biological tissue presents a direct indication of said i-th pathological condition PCᵢ of the biological tissue;
in response to user input about the i-th pathological condition to be inspected for in the biological tissue, selecting, in said assignment data, the respective i-th imaging mode data, and generating corresponding operational data to an imaging device to implement said imaging mode in at least one imaging session on said predetermined location and detect a combined spectral response of said location to the illumination pattern, and generate corresponding image data indicative of the detected combined spectral response; and
processing the image data and providing output data about said i-th pathological condition.

20. The method of claim 19, wherein said detection of the combined spectral response is performed by a color camera device configured for concurrently detecting the plurality of auto-fluorescent responses of different emission wavelengths.

21. The method of claim 20, wherein said detection comprises collecting the combined spectral response by a pixel matrix defining multiple detection channels of different colors, such that the image data is indicative of different detected wavelengths of said combined spectral response and corresponding location data in the biological tissue where said different detected wavelengths are originated.

22. The method of claim 19, wherein said processing of the image data comprises identifying, in said image data, image data pieces corresponding to the different wavelengths contained in said detected combined spectral response, and determining a relation between said image data pieces to identify the corresponding location data in the biological tissue where said different detected wavelengths are originated.

23. The method of claim 19, wherein the illumination pattern includes one or more visualization wavelengths selected to enhance imaging of blood vessels in vicinity of the location being imaged, such that the image data includes a combined image of the blood vessels and said two or more excited substances.

24. The method of claim 23, wherein said one or more visualization wavelengths includes one or more exciting wavelengths selected to excite substances associated with blood vessel structures.

25. The system of claim 24, wherein said one or more visualization wavelengths includes at least one of 436 nm, 517 nm and 660 nm wavelengths.

26. The system of claim 25, wherein the illumination pattern includes 350 nm wavelength for exciting elastin and collagen substances.

27. The method according to claim 19, wherein said assignment data comprises data indicative of pathological condition relating to diabetic retinopathy (DR) assigned with the one or more imaging mode to be performed on the location in a retina region utilizing comprising at least one of the following:
(a) the illumination pattern comprising the exciting wavelength range comprising 385 nm and 400 nm wavelengths selected to induce simultaneous auto-fluorescence response of AGEs and plasma substances,
(b) the illumination pattern comprising the exciting wavelength range comprising 365 nm and 430 nm wavelengths selected to induce simultaneous auto-fluorescence response of NADH and flavoproteins substances.

28. The method according to claim 19, wherein said assignment data comprises data indicative of pathological condition relating to at least one of the following:
macular degeneration (AMD) assigned with the one or more imaging mode to be performed on the location in a retina region comprising the exciting wavelength range including 350 nm wavelength selected to induce simultaneous auto-fluorescence responses of amyloid and fatty acids substances; and
proliferative diabetic retinopathy (DR) assigned with the one or more imaging mode to be performed on the location in a cornea region comprising the exciting wavelength range including 385 nm and 430 nm wavelengths selected to induce simultaneous auto-fluorescence responses of AGE and flavoproteins substances.

29. A method for inspecting a biological tissue for one or more pathological conditions, the method comprising:
illuminating a region within the biological tissue by an illumination pattern comprising a predetermined exciting wavelength range comprising a predetermined number N (N≥1) of predetermined exciting wavelengths selected to penetrate through said region of the biological tissue and interact with different layers of the biological tissue in said region along a propagation path of the illumination pattern in said region to thereby induce auto-fluorescent response of one or more predetermined biological substances of types naturally existing in biological tissues; and detecting spectral response of the biological tissue to said illumination pattern during said propagation of the illumination pattern; and
analyzing data indicative of the spectral response, and upon identifying in said detected spectral response intensity variation of emission wavelengths of said one or more substances, determining optimal focal length of the illumination pattern with respect to excitation of said one or more biological substances, and generating corresponding operational data comprising focal data for use in further measurements to optimize detection of existence and spatial distribution of said one or more predetermined biological substances in the tissue region, being indicative of the one or more pathological conditions.
